(19) United States
(12) Patent Application Publication
(10) Pub. No.: US 2010/0063292 A1
(43) Pub. Date: Mar. 11, 2010

(54) PROCESS FOR THE PREPARATION OF TRIFLUOROETHOXYTOLUENES.

(75) Inventors: Daina Zicane, Riga (LV); Janis Jaunbergs, Riga (LV)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Grindeks A Joint Stock Company, Riga (LV)

(21) Appl. No.: 12/312,323

(22) PCT Filed: Nov. 2, 2007
(86) PCT No.: PCT/EP2007/061818

§ 371 Date: May 5, 2009

(30) Foreign Application Priority Data
Nov. 6, 2006 (EP) 06123547.9

(51) Int. Cl. C07D 211/32; C07C 43/164

(52) U.S. Cl. 546/233; 568/626

(57) ABSTRACT

The present invention relates to an process for the preparation 2,5-bis(2,2,2-trifluoroethoxy)toluene [II]. The compound 2,5-bis(2,2,2-trifluoroethoxy)toluene is useful as intermediate for pharmaceutical industry, especially useful as an intermediate for the preparation of Flecainide and pharmaceutically acceptable salts.

### TECHNICAL FIELD

The present invention relates to an novel process for the preparation a substituted 1,4-bis(2,2,2-trifluoroethoxy)benzene of the formula (I)

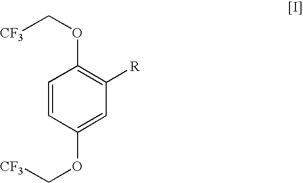

wherein R is methyl

2,5-bis(2,2,2-trifluoroethoxy)toluene of the formula [II] is useful as a novel intermediate in the pharmaceutical industry.

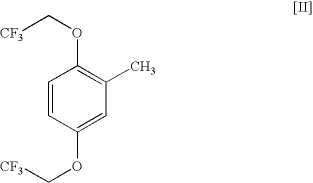

For example use as novel intermediate for the synthesis of the antiarrhythmic drug-Flecainide [III] and pharmaceutically acceptable salts thereof.

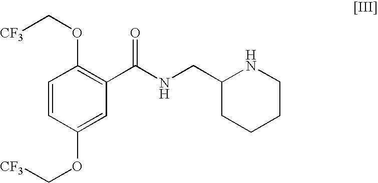

### BACKGROUND ART

2,5-bis(2,2,2-trifluoroethoxy)toluene is a derivative of 1,4-bis(2,2,2-trifluoroethoxy)benzene, which as well is used as intermediate for synthesis of Flecainide.

It is known that 1,4-bis(2,2,2-trifluoroethoxy)benzene can be obtained by the reaction of 1,4-dibromobenzene with 2,2,2-trifluoroethanol in the presence of sodium hydride, N,N-dimethylformamide and cuprous iodide GB 2045760 (RIKER LABORATORIES) 1980.03.18.

A serious disadvantage of using 1,4-dibromobenzene to form 1,4-bis(2,2,2-trifluoroethoxy)benzene is that the process requires the reaction of 8 equivalents of 2,2,2-trifluoroethanol while only 2 equivalents are theoretically needed. The use of less than 8 equivalents of 2,2,2-trifluoroethanol results in incomplete conversion to 1,4-bis(2,2,2-trifluoroethoxy)benzene, with the starting material and 1-bromo-4-(2,2,2-trifluoroethoxy)benzene as the main impurities. Isolation and purification of desired product from this mixture is not practical on an industrial scale.

Another method involves the process to obtained the 1,4-bis(2,2,2-trifluoroethoxy)benzene (III) is by the reaction of 4-fluoro-1-bromobenzene with 2,2,2-trifluoroethanol in the presence of sodium hydride, N,N-dimethylformamide and CuBr₂ at about 100-105° C. WO 02/066413 (NARCHEM CORPORATION) 2002.02.20.

As disadvantage of this process is that the system NaH with the N,N-dimethylformaide has a high safety risk for large-scale industrial synthesis since this system can decompnose exothermically in an uncontrollable manner. BUCKLEY, J. Report on thermal reaction. Chem. Eng. News. 1982, vol. 60, no. 28, p. 5., POND, D. Sodium hydride and DMF. Chem. Eng. News. 1982, vol. 60, no. 37, p. 5,43.

Both of these processes involve three steps to obtain the main intermediate 2,5-bis(2,2,2-trifluoroethoxy)benzoic acid, for preparation Flecainide. Disadvantage of multi steps process disclosed in that, what yield of 2,5-bis(2,2,2-trifluoroethoxy)benzoic acid is reducing.

### DISCLOSURE OF INVENTION

The above object is attained by present invention which provides a novel process for the preparation of 2,5-bis(2,2,2-trifluoroethoxy)toluene of the formula [II]

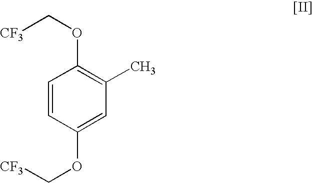

which process comprises reacting a 2,5-dihalotoluene of formula [IV]

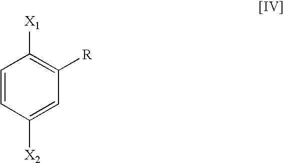

wherein X₁ and X₂ is halogen, the X₁ and X₂ substituents may be the same or different, R is methyl.

With 2,2,2-trifluoroethanol in the presence alkali metal and a copper containing material.

The process of preparation of 2,5-bis(2,2,2-trifluoroethoxy)toluene by using as starting material 2,5-dihalotolunes has a following advantages:

1. use of metallic sodium instead of sodium hydride and as dipolar aprotic solvent using N,N-dimethylformamide is attempt safer method in industrial production;
2. production of desired final product in high yields;
3. amenability for large scale production which does not require specialized equipment.

The method of the present invention involves the novel initial preparation of 2,5-bis(2,2,2-trifluoroethoxy)toluene by reaction 2,5-dihalotoluene with 2,2,2-trifluoroethanol in the presence alkali metal and copper containing material. The 2,2,2-trifluoroathanol reacts in about 2-10 fold molar excess of the 2,5-dihalotoluene to replace the 2,5-dihalotoluene aromatic halogen substituents with trifluoroethoxy groups. Bases that enable the reaction include alkali metals, e.g., metallic sodium. A copper containing material is used as a catalyst in the reaction, e.g., copper (II) sulphate. For performing reaction N,N-dimethylformamide can be used as an dipolar aprotic solvent. The best mode for carrying out the reaction is a temperature of about 85 to 105° C.

As it is mentioned above 2,5-bis(2,2,2-trifluoroethoxy)toluene can be used as a novel intermediate for preparation Flecainide and pharmaceutically acceptable salts in high yields up to 82%. The preparation of Flecainide can be released by a sequential combination of four process steps, starting from 2,5-dibromotoluene:
1. replacement of the 2,5-dibromotoluene aromatic bromine substituents by trifluoroethoxy groups, and, thus, preparation of 2,5-bis(2,2,2-trifluoroethoxy)toluene or any of the intermediate substitution products 2-bromo-5-trifluorethoxytoluene and 5-bromo-2-trifluorethoxytoluene,
2. oxidation of 2,5-bis(2,2,2-trifluoroethoxy)toluene with potassium or sodium permanganate, to produce 2,5-bis(trifluorethoxy)benzoic acid,
3. activation of 2,5-bis(2,2,2-trifluorethoxy)benzoic acid in-situ with a chloroformate, and substitution of the leaving carbonate group with 2-(aminomethyl)pyridine, and
4. hydrogenation of 2,5-bis(2,2,2-trifluorethoxy)-N-(pyrid-2-yl-methyl)benzamide to obtain Flecainide acetate.

### BEST MODE FOR CARRYING OUT THE INVENTION

The present invention will be described in more detail with the aid of the following examples, which are merely representative and should not serve to limit the scope of the invention.

### Preparation of 2,5-bis(2,2,2-trifluoroethoxy)toluene from 2,5-dibromotoluene

2,2,2-trifluoroethanol (55.0 g, 0.550 mol) was added to dioxane (125 mL) in a glass vessel fitted with a reflux condenser. Sodium metal (11.5 g, 0.500 mol) was added in portions of 2-3 grams to the solution, resulting in a temperature increase from 22° C. to 90° C. The solution was stirred at 85-105° C. until the sodium dissolution was completed, then N,N-dimethylformamide (100 mL) was added, followed by 2,5-dibromotoluene (I) (42.5 g, 0.170 mol) and anhydrous copper (II) sulphate (2.9 g, 0.018 mol). The reaction mixture was stirred at 95-100° C. for 4 hours, and then cooled to 25-30° C. and poured into 900 ml of a cold (5-10° C.) 40% aqueous methanol solution.

Concentrated hydrochloric acid was added (˜25 mL, 0.300 mol), until pH=1-2. The crystallization suspension was stirred for 1 hour at −5 to 0° C., the solid white precipitate was filtered, after which the reaction vessel and the product cake on the filter were rinsed with 50 mL of water. The intermediate 2,5-bis(2,2,2-trifluoroethoxy)toluene (II) was dried at ambient temperature and pressure for 5 hours, then at 22 to 24° C. and at a reduced pressure for 4 hours. The 2,5-bis(2,2,2-trifluoroethoxy)toluene was obtained as a white or off-white powder (45.5 g, 92%) having a melting point of 37° C. to 42° C.

### INDUSTRIAL APPLICATION

The 2,5-bis(2,2,2-trifluoroethoxy)toluene [II] can be used as intermediate in pharmaceutical industry. For example, can be used as useful intermediate for obtaining Flecainide [III] and pharmaceutically acceptable salts. By using 2,5-bis(2,2,2-trifluoroethoxy)toluene as intermediate yield of Flecainide [III] and pharmaceutically acceptable salts can be increased to 86%.

1-11. (canceled)

12. A substituted 1,4-bis(2,2,2-trifluoroethoxy)benzene of the formula [I] 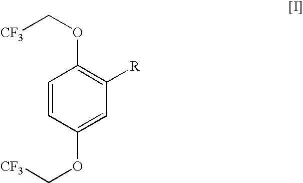
where R is methyl.

13. The compound of claim 12, which is 2,5-bis(2,2,2-trifluoroethoxy)toluene.

14. A process for the preparation of 2,5-bis(2,2,2-trifluoroethoxy)toluene of formula [II] 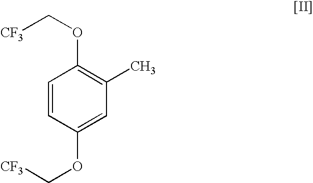
which process comprises reacting a 2,5-dihalotoluene of the formula [IV] 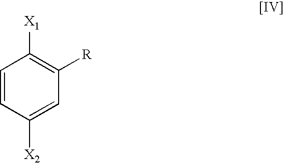
wherein X₁ and X₂ represent halogen, and the X₁ and X₂ substituents may be the same or different, and R is methyl, with 2,2,2-trifluoroethanol in the presence of an alkali metal and a copper containing material.

15. The process of claim 14, wherein X₁ and X₂ are Br, and the compound of formula [IV] is 2,5-dibromotoluene.

16. The process of claim 14, wherein the reaction is carried out in the presence of copper (II) sulphate.

17. The process of claim 14, wherein the 2,2,2-trifluoroethanol is reacted in a first step with a strong base to form a 2,2,2-trifluoroethoxide, which is reacted in a second step with a 2,5-dihalotoluene of formula [IV] in the presence of copper containing material.

18. The process of claim 14, wherein the alkali metal is metallic sodium.

19. The process of claim 14, wherein the reaction is conducted is an aprotic solvent.

20. The process of claim 14, wherein the aprotic solvent is a dipolar aprotic solvent or an N-containing heterocycle or mixtures thereof.

21. The process of claim 20, wherein the dipolar apotic solvent is N,N-dimethylformamide.

22. The process of claim 14, wherein the molar ratio between the 2,5-dihalotoluene and 2,2,2-trifluoroethanol is from 1:2 to 1:10.

23. The process of claim 21, wherein the molar ratio between the 2,5-dihalotoluene and 2,2,2-trifluoroethanol is 1:6.
